(19) United States
(12) Patent Application Publication
(10) Pub. No.: US 2008/0181933 A1
(43) Pub. Date: Jul. 31, 2008

(54) Chewing Gum Compositions of Varenicline

(75) Inventors: Barbara Alice Johnson, Niantic, CT (US); Carl Bernard Ziegler, East Lyme, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: PFIZER INC.

(21) Appl. No.: 11/816,731

(22) PCT Filed: Mar. 9, 2006
(86) PCT No.: PCT/IB06/00735

§ 371 Date: Aug. 21, 2007

Related U.S. Application Data
(60) Provisional application No. 60/664,094, filed on Mar. 21, 2005.

(51) Int. Cl. A61K 9/68; A23G 4/06; A23G 4/18; A61K 31/4985; A23G 4/20

(52) U.S. Cl. 424/440; 426/3; 426/5; 424/48; 514/250

(57) ABSTRACT

A chewing gum composition including a water insoluble base portion; a water soluble portion; and a therapeutically effective amount of varenicline or its pharmaceutical acceptable salt thereof. A method for reducing nicotine addiction and aiding in the cessation or lessening of tobacco use in an individual by administering to an oral cavity of an individual a chewing gum composition including an effective amount of varenicline or its pharmaceutical acceptable salt thereof; and chewing the gum composition to cause the varenicline or its pharmaceutical acceptable salt thereof to be released from the chewing gum composition into the oral cavity of the individual. A method of manufacturing a chewing gum composition.

### FIELD OF THE INVENTION

The present invention relates to pharmaceutical compositions for medicinal uses thereof.

### BACKGROUND ART

Varenicline has the structure:

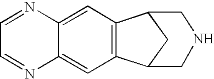

Varenicline is also known as 5,8,14-triazatetracyclo[10.3.1.0^{2,11}0.0^{4,9}]-hexadeca-2(11),3,5,7,9-pentaene or 7,8,9,10-tetrahydro-6,10-methano-6H-pyrazino[2,3-h][3]-benzazepine. Varenicline and pharmaceutically acceptable acid addition salts thereof are referred to in International Patent Publication WO 99/35131, published Jul. 15, 1999, the contents of which are incorporated herein by reference.

Varenicline binds to neuronal nicotinic acetylcholine specific receptor sites and is useful in modulating cholinergic function. Accordingly, this compound is useful in the treatment of various conditions or diseases including, but not limited to, inflammatory bowel disease (including, but not limited to, ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome.

Varenicline is a highly potent compound such that dosage forms are necessarily highly diluted with excipients. The excipients provide dosage forms with adequate stability, while also providing for such desirable features as controlling the drug dissolution (e.g., either fast dissolving or slow dissolving in a controlled-release system as described in co-pending applications U.S. Patent Publication No. 2003-0180360 A1, published Sep. 25, 2003, and Ser. No. 10/848,464, filed May 18, 2004, the contents of which are hereby incorporated by reference in their entirety), masking bad taste, and providing appropriate properties for preparation of the dosage form (i.e., compression properties for tablets). Finally, because of the high dilution with excipients, reactivity of varenicline with the excipients themselves or with trace impurities (i.e., degradants) of the excipients can be especially problematic.

There are advantages of delivering varenicline in the form of a chewing gum composition. For example, relative to an oral dosage form such as a tablet or capsule delivery of varenicline via chewing gum composition allows for a portion of the dose to be rapidly absorbed through the buccal and sublingual routes. The remainder of the dose would then be dispersed and/or dissolved in the saliva and then swallowed ultimately being absorbed via the gastrointestinal route. Chewing gum compositions of varenicline would be a preferred choice by patients who have difficulty in swallowing tablets, capsules or other solids.

The tablet dosage form of varenicline has shown, in some instances, a certain level of nausea in patients. There is a need to reduce these side effects. A gradual release of the varenicline dosage form such as would be the case from a chewing gum composition might prove to be useful towards reducing the incidence of nausea and enhance the desirability of the drug to a larger patient population requiring its use.

Accordingly, there is a need for providing chewing gum dosage forms of varenicline.

### SUMMARY OF THE INVENTION

The present invention provides a chewing gum composition of varenicline or its pharmaceutically acceptable salt. The solid dosage form of varenicline is present within the chewing gum composition. By chewing the gum composition of the present invention, the drug is gradually released from the dosage form thereby allowing a portion of the free drug to be absorbed via the buccal and sublingual absorption routes or it is swallowed and enters the systemic system via the gastrointestinal route. This gradual release of varenicline or its pharmaceutically acceptable salt by the chewing gum composition of the present invention may reduce or eliminate the nausea side effect associated with the drug.

In one embodiment of the composition, the chewing gum is chewed for at least 2 minutes.

In an embodiment of the composition, the chewing gum contains varenicline tartrate.

In another embodiment of the composition, the particle size of varenicline, preferably varenicline tartrate, powder incorporated into the chewing gum composition is approximately 0.1 microns to about 200 microns in diameter, more preferably from about 0.1 microns to about 50 microns in diameter.

In another embodiment of the composition, each piece of the chewing gum contains approximately 1 mg to about 10 mg of pure varenicline tartrate, and an insoluble gum base and at least one other ingredient chosen from the list comprising gum bases, fillers, texturizers, softeners, emulsifiers, sweeteners, flavor masking agents, colorants, and flavoring agents.

In another embodiment of the composition, the chewing gum product is comprised of a core of chewing gum and the active ingredient.

In another embodiment of the composition, the chewing gum can comprise the core which is substantially enclosed by an outer-coating or shell containing the active ingredient.

In another embodiment of the composition, a compressible excipient, containing the active ingredient is tableted and then located within a coating or shell consisting of a chewing gum product.

In another embodiment of the composition, a compressible excipient, containing a portion of the active ingredient is tableted and then located within a coating or shell consisting of a chewing gum and the remaining portion of the dose of active ingredient.

In another embodiment, the present invention provides a method for reducing nicotine addiction, aiding in the cessation of, or lessening of, tobacco use in a subject.

### DETAILED DESCRIPTION OF THE INVENTION

Generally, the present invention provides chewing gum compositions and related methods for systemic absorbtion of varenicline or its pharmaceutically acceptable salts.

The present invention utilizes varenicline or its pharmaceutically acceptable salt as the active ingredient. Varenicline can be used per se or in the form of its pharmaceutically acceptable salt, solvate and/or hydrate. Although any pharmaceutically acceptable form of varenicline can be used in connection with the present invention, it is preferable to use a salt form of the drug. A particularly preferred salt form of the drug is the L-tartrate salt.

In particular, the present invention provides a method for reducing nicotine addiction or aiding in the cessation or lessening of tobacco use in a subject. The method includes steps of administering to a subject an amount of the varenicline that is effective in reducing nicotine addiction or aiding in the cessation or lessening of tobacco use via administration of a chewing gum dosage form of the drug.

The present invention can be used to treat disorders or conditions including, but not limited to, inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions; dependencies on, or addictions to, nicotine, tobacco products, alcohol, benzodiazepines, barbiturates, opioids or cocaine; headache, stroke, traumatic brain injury (TBI), obsessive-compulsive disorder (OCD), psychosis, Huntington's Chorea, tardive dyskinesia, hyperkinesia, dyslexia, schizophrenia, multi-infarct dementia, age related cognitive decline, epilepsy, petit mal absence epilepsy, senile dementia of the Alzheimer's type (AD), Parkinson's disease (PD), attention deficit hyperactivity disorder (ADHD), Tourette's Syndrome, and any other similar disorder or condition known to those of skill in the art.

The term “buccal absorption” as used herein means a method for drug absorption through the buccal (i.e., inner cheek) tissue.

The term “sublingual absorption” as used herein means delivery of the active compound of the present invention across any tissue under the tongue.

The term “transmucosal absorption” as used herein means delivery of the active compound of the present invention across any mucosal membrane.

The term “varenicline,” as used herein means the drug that binds to neuronal nicotinic acetylcholine specific receptor sites, and is useful in modulating cholinergic function. Varenicline has the general formula of:

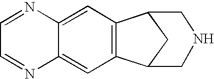

Varenicline includes the parent drug and all pharmaceutically acceptable salts and prodrugs thereof. The parent drug of varenicline is described in International Patent Publication WO 99/35131, published Jul. 15, 1999, the contents of which are incorporated herein by reference in their entirety. In any of the embodiments, varenicline or any of its pharmaceutically acceptable salts, solvates and/or hydrates can be used. Procedures for making varenicline are described in U.S. Pat. No. 6,410,550, the contents of which are incorporated herein by reference in their entirety. The resolution of racemic mixtures of varenicline is described in WO01/62736, which is also incorporated herein by reference in its entirety.

The term “mgA” refers to the number of milligrams of active drug based on the free base form of the drug.

The term “substantially reducing carbohydrate-free” as used herein means less than approximately 20 w/w % of a reducing carbohydrate (including, but not limited to, lactose). Preferably, dosage forms prepared in accordance with the present invention contain less than 10 w/w % of a reducing carbohydrate, and more preferably, less than 5 w/w %.

The term “pharmaceutically acceptable” means the substance or composition must be compatible chemically, physically, and/or toxicologically, with the other components comprising a formulation, and/or the patient being treated therewith.

The term “pharmaceutically acceptable salt” means non-toxic acid addition salts derived from inorganic and organic acids. Suitable salt derivatives include, but are not limited to, halides, thiocyanates, sulfates, bisulfates, sulfites, bisulfites, arylsulfonates, alkylsulfates, phosphonates, monohydrogen-phosphates, dihydrogenphosphates, metaphosphates, pyrophosphonates, alkanoates, cycloalkylalkanoates, arylalkonates, adipates, alginates, aspartates, benzoates, fumarates, glucoheptanoates, glycerophosphates, lactates, maleates, nicotinates, oxalates, palmitates, pectinates, picrates, pivalates, succinates, tartarates, citrates, camphorates, camphorsulfonates, digluconates, trifluoroacetates, and the like.

The term “active ingredient” means a therapeutically active compound (i.e., varenicline) as well as any prodrugs thereof and pharmaceutically acceptable salts, hydrates, and solvates of the compound and the prodrugs.

The term “other ingredients ” means any excipients, diluents, binders, lubricants, glidants, carriers, surfactants, fillers, texturizers, softeners, emulsifiers, sweeteners, flavors, colorants and mixtures thereof that are formulated with varenicline or any pharmaceutically acceptable salts, hydrates, and solvates of this drug

The term “appropriate period of time” or “suitable period of time” means the period of time necessary to achieve a desired effect or result. For example, a mixture can be blended until a potency distribution is reached that is within an acceptable range for a given application or use of the blended mixture.

The term “unit dose,” “unit dosage,” or “unit dosage form” means a physically discrete unit that contains a predetermined quantity of active ingredient calculated to produce a desired therapeutic effect. The chewing gum dosage form can be in the form including, but not limited to, tablets, lozenge , a stick, slab, pellets, squares, balls or other unitary structure and other forms known to those of skill in the art.

The term “effective amount,” as used herein means the amount determined by such considerations as are known in the art of reducing nicotine addiction or aiding in the cessation or lessening of tobacco use in an individual, wherein it must be effective to provide measurable relief in treated individuals such as exhibiting improvements including, but not limited to, more rapid recovery, improvement or elimination of symptoms or reduction of complications, lack of dependency upon nicotine-containing compounds, lack of desire towards nicotine-containing compounds, or other measurements as appropriate and known to those skilled in the medical arts.

The present invention has numerous embodiments. In any of the embodiments, pharmaceutical compositions of varenicline can be desirably administered in doses ranging from about 0.1 mgA up to about 6 mgA per day (where mgA refers to mg of active drug based on the free base form of the drug), more preferably from about 0.5 to 4 mgA/day, and most preferably from about 1 to 4 mgA per day in single or divided doses. Variations in such dosages, however, necessarily occur depending upon the weight and condition of the subject being treated. Depending on individual responses, dosage levels below the lower limit of the aforesaid range can be more than adequate, while in other cases still larger doses can be employed without causing any harmful side effects. The final pharmaceutical composition is processed into a unit dosage form and then packaged for distribution. The processing steps vary depending upon the particular chewing gum dosage form. Those of skill in the art are well aware of the procedures used for manufacturing the various unit dosage forms.

The preferred formulations of the present invention contain less than about 20% wt reducing carbohydrates. The presence of reducing carbohydrates is detrimental to the drug stability on storage. Reducing carbohydrates are sugars and their derivatives that contain a free aldehyde or ketone group capable of reaction with varenicline's secondary amine. Examples of reducing carbohydrates include, but are not limited to, monosaccharides, disaccharides, lactose, glucose, fructose, maltose, and other similar sugars known to those of skill in the art.

The present invention provides chewing gum compositions containing varenicline and its pharmaceutically acceptable salts, herein referred to as the active ingredient and methods for delivering it to an individual. Accordingly, the chewing gum composition is chewed for at least two minutes. The active ingredient is gradually released from the composition and into the saliva of the oral cavity. During continual chewing, a major portion of the active ingredient is released into the saliva within about 30 minutes During further continual chewing, essentially the entire active ingredient is released into the saliva within about 1 hour. Some of the active ingredient is absorbed through the oral mucosa of the oral cavity into the systemic circulation via the buccal or sublingual absorption routes and a portion of the drug of the drug absorption occurs via the gastrointestinal route.

Moreover, it is reasonable to assume that the active ingredient within a chewing gum composition, is gradually released from the chewing gum over a time period of up to about an hour. It is believed that the gradual release of the active ingredient from the chewing gum composition prevents excessive concentrations of the drug from developing within the stomach, the gastrointestinal tract and the blood which in turn reduces or prevents the nausea side effect.

Preferably, the form of active ingredient contained within the chewing gum formulations is varenicline tartrate. The effective amount of varenicline tartrate contained within the chewing gum compositions is approximately 0.1 mg to 10 mg of pure varenicline tartrate per piece of chewing gum. For example, a typical piece of chewing gum according to the present invention weighs about 1 to about 3 grams total and would contain approximately 0.1 mg to about 10 mg of pure varenicline tartrate as a portion of that particular total. Thus, one serving of varenicline tartrate would be one piece of the chewing gum composition.

The active ingredient of the present invention can be contained in a variety of different chewing gum compositions as cited in the US Patent references: U.S. Pat. No. 6,627,234; U.S. Pat. No. 6,586,023; U.S. Pat. No. 6,602518; U.S. Pat. No. 6,592,850; U.S. Pat. No. 6,613,346; U.S. Pat. No. 6,558,692; U.S. Pat. No. 6,531,114; U.S. Pat. No. 6,465,003; U.S. Pat. No. 6,426,090; U.S. Pat. No. 6,355,265; U.S. Pat. No. 6,350,480; U.S. Pat. No. 6,322, 806; U.S. Pat. No. 6,290,985 and these are hereby incorporated as reference.

The chewing gum can be a number of different structures. For example, the chewing gum can be a single piece, for example, a stick, slab, or other unitary structure.

On the other hand, the chewing gum can comprise an outer-coated formulation. In this regard, if desired, the active ingredient can be located within a coating or shell that substantially encloses a gum center. Alternatively, the active ingredient may be located within the gum center. Or the active ingredient may be located in both the coating or shell and the gum center. The coating can comprise, in an embodiment, approximately 20 to about 75% of the chewing gum composition. In addition to the active ingredient, the coating can include a masking agent to improve the taste of the coating containing the active ingredient. A variety of masking agents can be utilized including: sucralose; zinc gluconate; ethyl maltol; glycine; acesulfame-k; aspartame; saccharin; fructose; xylitol; spray dried licorice root; glycerrhizine; dextrose; sodium glutonate; glucono delta-lactone; ethyl vanillin; vanillin; normal and high potency sweeteners; and a variety of appropriate flavors. A sufficient masking agent is used to mask the taste of the active ingredient. If desired, more than one masking agent can be used.

A variety of methods can be used for creating a coated chewing gum. For example, the coating can be applied in a three phase operation to a chewing gum center. In the first phase, a crude coating of syrup and active ingredient is applied to the center. This is followed by a second phase called the finishing coating in which a fine powder and longer tumbling is used to produce a smooth finish. Finally, a shellacking and polishing third phase is performed to provide a high sheen, smooth finish. If desired, the second and third phases can be eliminated. The coating can surround a variety of different types of gum center compositions as set forth below.

In another embodiment of the present invention, a compressible excipient is tableted and then coated with a chewing gum product including the active ingredient. The tableted excipient can comprise, by way of example and not limitation, dextrose, sucrose, or other saccharides, sorbitol, mannitol, isomalitol, other compressible sugar alcohols or combinations thereof. The tableted compressible excipient is substantially surrounded by a gum coating. The coating includes the active ingredient and, in an embodiment, the coating comprises at least 50% by weight of the product. Additionally, the coating can include a taste masking agent, an opacifier.

Alternatively, a mixture of a compressible excipient and the active ingredient is tableted and then coated with a chewing gum product which may optionally contain the active ingredient. The tableted excipient can comprise, by way of example and not limitation, dextrose, sucrose, or other saccharides, sorbitol, mannitol, isomalitol, other compressible sugar alcohols or combinations thereof. The tableted compressible excipient is substantially surrounded by a gum coating. The coating includes the active ingredient and, in an embodiment, comprises at least 50% by weight of the product. Additionally, the coating can include a taste masking agent, an opacifier. Referring now to the chewing gum of the present invention, the chewing gums can be low or high moisture, sugar or sugarless, wax containing or wax free, low calorie (via high base or low calorie bulking agents), and/or may contain other dental and/or medicinal agents.

In general, a chewing gum typically comprises a water-soluble bulk portion, a water-insoluble chewable gum base portion, and a flavoring agent. The water-soluble portion dissipates with a portion of the flavoring agent over a period of time during chewing. The gum base portion is retained in the mouth throughout the chew. The term chewing gum refers to both a chewing and bubble gum in its general sense.

The insoluble gum base generally comprises elastomers, resins, fats and oils, softeners and inorganic fillers. The gum base may or may not include wax. The insoluble gum base can constitute approximately 5% to about 95% by weight of the chewing gum, more commonly the gum base comprises 10% to about 50% of the gum, and in some preferred embodiments approximately 15% to about 35%, by weight, of the chewing gum.

In an embodiment, the chewing gum base of the present invention contains about 20% to about 60% by weight synthetic elastomer, about 0% to about 30% by weight natural elastomer, about 5% to about 55% by weight elastomer plasticizer, about 4% to about 35% by weight filler, about 5% to about 35% by weight softener, and optional minor amounts (about 1% or less by weight) of miscellaneous ingredients such as colorants, antioxidants, flavoring agents, etc. Elastomers provide the rubbery, cohesive nature of the gum, which varies depending on this ingredient's chemical structure and how it is compounded with other ingredients.

Synthetic elastomers may include, but are not limited to, polyisobutylene, isobutylene-isoprene copolymer (butyl rubber), styrene-butadiene, copolymers having styrene-butadiene ratios of about 1:3 to about 3:1, polyvinyl acetate, vinyl acetate vinyl laurate copolymer having a vinyl laurate content of about 5% to about 50% by weight of the copolymer, and combinations thereof. Natural elastomers may include natural rubber such as smoked or liquid latex and guayule as well as natural gums such as jelutong, lechi caspi, perillo, sorva, massaranduba balata, massaranduba chocolate, nispero, rosindinha, chicle, gutta hang kang, and combinations thereof. The preferred synthetic elastomer and natural elastomer concentrations vary depending on whether the chewing gum in which the base is used is adhesive or conventional, bubble gum or regular gum. Preferred natural elastomers include jelutong, chicle, sorva and massaranduba balata.

Elastomer plasticizers may include, but are not limited to, rosin esters such as glycerol esters of rosin, methyl esters of rosin, pentaerythritol esters of rosin; terpene resins derived from alpha-pinene, beta-pinene, and/or d-limonene; and any suitable combinations of the foregoing. The resin tackifiers regulate the cohesiveness and tackiness of the final gums. The preferred elastomer plasticizers will also vary depending on the specific application, and on the type of elastomer which is used.

Fillers/texturizers may include magnesium and calcium carbonate, ground limestone, silicate types such as magnesium and aluminum silicate, clay, alumina, talc, titanium oxide, mono-, di- and tri-calcium phosphate, cellulose polymers, such as wood, and combinations thereof. Fillers modify the texture of the gum base. The fillers can also be organic powders such as polyethylene, oat fiber, wood fiber, apple fiber, zein, gluten, gliadin, casein, and the like. Active ingredient powder can be added as a filler during base making to achieve better encapsulation which may result in longer active ingredient release.

Softeners/emulsifiers may include tallow, hydrogenated tallow, hydrogenated and partially hydrogenated vegetable oils, cocoa butter, glycerol monostearate, glycerol triacetate, lecithin, non-hydrogenated, partially hydrogenated and fully hydrogenated mono-, di- and tri-glycerides from cottonseed, soybean, palm, palm kernel, coconut, and safflower sources, and other medium chain triglycerides, acetylated monoglycerides, fatty acids (e.g. stearic, plasmatic, oleic and linoleic acids), and combinations thereof. Such softeners/emulsifiers modify the texture of the gum base by introducing sharp melting transition during chewing.

Colorants and whiteners may include FD&C-type dyes and lakes, fruit and vegetable extracts, titanium dioxide, and combinations thereof. Colorants impart characteristics and remove or mask undesired characteristics in the chewing gum formulation.

The gum base may or may not include wax. An example of a wax-free gum base is disclosed in U.S. Pat. No. 5,286,500, the disclosure of which is incorporated herein by reference. Waxes aid in the curing of gum bases and in improving shelf life and texture of the final gum product. Wax crystals also improve the release of flavor from the final product.

Such gum bases are typically prepared by adding an amount of the elastomer, resin tackifier or softener, and filler to a pre-heated sigma blade mixer maintaining a temperature of from about 50° F. to about 240° F. The initial amounts of ingredients comprising the initial mass of the insoluble gum base may be determined by the working capacity of the mixing kettle in order to attain a proper consistency and by the degree of compounding desired to break down and soften the elastomer. The longer the period of time compounding and use of lower molecular weight or softening point gum base ingredients, a lower viscosity and firmness will result in the final gum base.

In addition to a water insoluble gum base portion, a typical chewing gum composition includes a water soluble bulk portion and one or more flavoring agents. The water soluble portion can include bulk sweeteners, high intensity sweeteners, flavoring agents, softeners, emulsifiers, colors, acidulants, fillers, antioxidants and other components that provide desired attributes.

Softeners are added to the chewing gum in order to optimize the chewability and mouth feel of the gum. The softeners, which are also known as plasticizers and plasticizing agents, generally constitute between approximately 0.5% to about 25% by weight of the chewing gum. The softeners may include glycerin, lecithin, and combinations thereof. Aqueous sweetener solutions such as those containing sorbitol, hydrogenated starch hydrolysates, corn syrup and combinations thereof, may also be used as softeners and binding agents in chewing gum.

Bulk sweeteners include both sugar and sugarless components. Bulk sweeteners typically constitute about 5% to about 95% by weight of the chewing gum, more typically, about 20% to about 80% by weight, and more commonly, about 30% to about 60% by weight of the gum. Sugar sweeteners generally include saccharide-containing components commonly known in the chewing gum art, including but not limited to, sucrose, dextrose, maltose, dextrin, dried invert sugar, fructose, levulose, glactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination.

High intensity artificial sweeteners can also be used, alone or in combination, with the above. Preferred sweeteners include, but are not limited to, sucralose, aspartame, salts of acesulfame, altitame, saccharin and its salts, cyclamic acid and its salts, glycerrhizinate, dihydrochalcones, thaumatin, monellin, and the like, alone or in combination. The range of these sweeteners in chewing gum formulations typically can range from about 0.02 to about 0.10% by weight for alitame, thaumatin and dihydrochalcones, and from about 0.1 to about 0.2% by weight for aspartame, sucralose, acesulfame and saccharin.

In order to provide longer lasting sweetness and flavor perception, it may be desirable to encapsulate or otherwise control the release of at least a portion of the artificial sweetener. Techniques such as wet granulation, wax granulation, spray drying, spray chilling, fluid bed coating, coacervation, and fiber extension may be used to achieve the desired release characteristics.

Combinations of sugar and/or sugarless sweeteners may be used in the chewing gum. Additionally, the softener may also provide additional sweetness such as with aqueous sugar solutions.

If a low calorie gum is desired, a low caloric bulking agent can be used. Examples of low caloric bulking agents include, but are not limited to: polydextrose; Raftilose®, Raftiline® (both available from Orafti Group, Tienen, Belgium); fructooligosaccharides (NutraFlora® available from GTC Nutrition LLC, Golden, Colo.); palatinose oligosaccharide; guar gum hydrolysate (Sun Fiber); or indigestible dextrin (Fibersol®-2 available from Matsutani Chemical Industry Co., Ltd., Hyogo, Japan). However, other low calorie bulking agents can also be used.

A variety of flavoring agents can be used, if desired. Flavoring agents like colorants are useful in chewing gum compositions to impart characteristics and to remove or mask undesired characteristics. In particular, the flavoring agent of the present invention should be capable of masking the unpleasant taste sensation associated with active ingredient. In doing so, the flavoring agent increases the contact time of the chewing gum composition of the present invention in the oral cavity. In doing so, the chewing gum composition enhances the absorption and bioavailability of the active ingredient component and prolongs the drug's therapeutic effects by gradually releasing the agent from the chewing gum composition.

The flavor can be used in amounts of about 0.1 to about 15% by weight of the gum, and preferably, about 0.2% to about 5% by weight. Flavoring agents may include essential oils, synthetic flavors or mixtures thereof including, but not limited to, oils derived from plants and fruits such as citrus oils, fruit essences, peppermint oil, spearmint oil, other mint oils, clove oil, oil of wintergreen, anise and the like. Artificial flavoring agents and components may also be used. Natural and artificial flavoring agents such as cocoa powder and heat-modified amino acids can be used as a flavoring agent within the present invention, and may be combined in any sensorially acceptable fashion.

The chewing gum composition of the present invention can be made utilizing manufacturing procedures known within the chewing gum arts. In general, chewing gum is manufactured by sequentially adding the various chewing gum ingredients to a commercially available mixer known in the art. After the initial ingredients have been thoroughly mixed, the gum mass is discharged from the mixer and shaped into the desired form such as by rolling into sheets and cutting into sticks, extruded into chunks or casting into pellets or balls.

Generally, the ingredients are mixed by first melting the gum base and adding it to the running mixer. The base may also be melted in the mixer itself. Color or emulsifiers may also be added at this time. A softener such as glycerin may also be added at this time, along with syrup and a portion of the bulking agent/sweetener. Further portions of the bulking agent/sweetener may then be added to the mixer thereafter. A flavoring agent is typically added with the final portion of the bulking agent/sweetener. A high-intensity sweetener is preferably added after the final portion of the bulking agent/sweetener and flavor have been added.

The entire mixing procedure typically takes from five to fifteen minutes, but longer mixing times may sometimes be required. Those skilled in the art will recognize that many variations of the above-described procedure may be followed.

In manufacturing the chewing gum composition of the present invention in particular, the active ingredient is mixed with the gum base, sweetener or sweetener mixture, and a flavoring agent. Preferably, the active ingredient is added early on in the mix. The smaller the amount of active ingredient used, the more necessary it becomes to preblend that particular ingredient to assume uniform distribution throughout the batch of gum.

Whether a preblend is used or not, in a preferred embodiment, the agent or active ingredient should be added within the first five minutes of mixing. Because the chewing gum composition of the present invention contains a water insoluble base, it enhances the gradual or controlled release of the active ingredient from the composition into the oral cavity. Thus, as the chewing gum composition of the present invention is chewed, the active ingredient component will gradually dissipate, along with the sweeteners and flavor, during chewing.

It is also preferable that the active ingredient of the present invention be first ground into fine particles to form a powder before being mixed with the gum base. The particle size of the active ingredient is preferably from about 0.1 microns to about 200 microns in diameter, more preferably from about 1 micron to about 50 microns in diameter.

In addition, prior to the active ingredient powder being mixed with the gum base, the fine powder is preferably dissolved into a liquid or liquid mixture, which is preferably water-insoluble. This is done to ease incorporation of the drug into the gum base and to enhance its uniform distribution throughout the overall chewing gum composition. Examples of liquid or liquid mixtures suitable for use within the present invention include, but are not limited to, an alcohol, an edible oil, glycerin, ethylene glycol, propylene glycol, triacetin, tributyrin glycerol mono- or di-stearate, acetylated mono-glyceride of coconut oil combinations thereof, and other like materials. The active ingredient powder can be mixed with molten or softened gum base directly, or it can be pre-mixed with a gum base ingredient such as polyvinyl acetate, rosin esters, polyterpene, waxes, fats, and the like.

In this invention, the active ingredient is used in the coating/panning of a pellet chewing gum. Pellet or ball gum is prepared as conventional chewing gum but formed into pellets that are pillow shaped, or into balls. The pellets/balls can be then sugar coated or panned by conventional panning techniques to make a unique coated pellet gum. The active agent may be soluble in flavor or can be blended with other powders often used in some types of conventional panning procedures. The active ingredient is isolated from other gum ingredients which modifies its release rate from chewing gum. The weight of the coating may be about 20% to about 50% of the weight of the finished product, but may be as much as 75% of the total gum product. The active ingredient will be based on the dosage for one or two pellets.

Conventional panning procedures generally coat with sucrose, but recent advances in panning have allowed use of other carbohydrate materials to be used in place of sucrose. Some of these components include, but are not limited to, dextrose, maltose, palatinose, xylitol, lactitol, hydrogenated isomaltulose, erythritol, maltitol, and other new alditols or combinations thereof. These materials may be blended with panning modifiers including, but not limited to, gum arabic, maltodextrins, corn syrup, gelatin, cellulose type materials like carboxymethyl cellulose or hydroxymethyl cellulose, starch and modified starches, vegetables gums like alginates, locust bean gum, guar gum, and gum tragacanth, insoluble carbonates like calcium carbonate or magnesium carbonate and talc. Antitack agents may also be added as panning modifiers, which allow the use of a variety of carbohydrates and sugar alcohols to be used in the development of new panned or coated gum products. Flavors may also be added with the sugar or sugarless coating and with the active to yield unique product characteristics. The active ingredient may be solubilized in ethanol or another suitable solvent and added with this type of film

Some film polymers can use water as the solvent in film coating.

Recent advances in polymer research and in film coating technology eliminates the problem associated with the use of solvents in coating. These advances make it possible to apply aqueous films to a pellet or chewing gum product. The active ingredient can be added to this aqueous film. This film may also contain a flavor along with a polymer and plasticizer. The active ingredient can also be dissolved in the aqueous or non-aqueous solvent and coated on the surface with the aqueous film. In some instances a combination of film and sugar or polyol coating may be useful, especially if the active is added with the film coating material. Also the film coating may be applied early, middle, or late in the coating process.

After a coating film with the active ingredient is applied to a chewing gum product, a hard shell sugar or polyol coating may then be applied over the film coated product. In some instances a soft shell sugar or polyol coating may also be used over the film coated product. The level of film coating applied to a pellet gum may be generally from about 0.5% to about 3% of the gum product. The level of overcoating of the hard or soft shell may be about 20% to about 75%. When the active ingredient is added with the film coating and not with the sugar/polyol coating, better control of the amount of the active ingredient in the product may be obtained. In addition, the sugar/polyol overcoating may give an improved stability to the active ingredient in the product.

The coating is initially present as a liquid syrup which contains from about 30% to about 80% or 85% of the coating ingredients previously described herein, and from about 15% or 20% to about 70% of a solvent such as water. In general, the coating process is carried out in a rotating pan.

Sugar or sugarless gum center tablets to be coated are placed into the rotating pan to form a moving mass.

The material or syrup which will eventually form the coating is applied or distributed over the gum center tablets. Flavoring agents may be added before, during and after applying the syrup to the gum centers. Once the coating has dried to form a hard surface, additional syrup additions can be made to produce a plurality of coatings or multiple layers of hard coating.

In a hard coating panning procedure, syrup is added to the gum center tablets at a temperature range of from about 100° F. to about 240° F. Mostly, the syrup temperature is from about 130° F. to about 200° F. throughout the process in order to prevent the polyol or sugar in the syrup from crystallizing.

The syrup may be mixed with, sprayed upon, poured over, or added to the gum center tablets in any way known to those skilled in the art.

In general, a plurality of layers is obtained by applying single coats, allowing the layers to dry, and then repeating the process. The amount of solids added by each coating step depends chiefly on the concentration of the coating syrup. Any number of coats may be applied to the gum center tablet.

Generally, no more than about 75-100 coats are applied to the gum center tablets. The present invention contemplates applying an amount of syrup sufficient to yield a coated comestible containing about 10% to about 75% coating. Where higher dosage of an active agent is needed, the final product may be higher than 75% coating.

Those skilled in the art will recognize that in order to obtain a plurality of coated layers, a plurality of premeasured aliquots of coating syrup may be applied to the gum center tablets. It is contemplated, however, that the volume of aliquots of syrup applied to the gum center tablets may vary throughout the coating procedure.

Once a coating of syrup is applied to the gum center tablets, the present invention contemplates drying the wet syrup in an inert medium. A preferred drying medium comprises air. Forced drying air contacts the wet syrup coating in a temperature range of from about 70° F. to about 115° F.

Generally, the drying air is in the temperature range of from about 80° F. to about 100° F. The invention also contemplates that the drying air possess a relative humidity of less than about 15 percent. Preferably, the relative humidity of the drying air is less than about 8 percent. The drying air may be passed over and admixed with the syrup coated gum centers in any way commonly known in the art. Generally, the drying air is blown over and around or through the bed of the syrup coated gum centers at a flow rate, for large scale operations, of about 2800 cubic feet per minute.

If lower quantities of material are being processed, or if smaller equipment is used, lower flow rates would be used. It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

Flavors are added to a sugar coating of pellet gum to enhance the overall flavor of gum. These flavors include spearmint flavor, peppermint flavor, wintergreen flavor, and fruit flavors. These flavors are generally preblended with the coating syrup just prior to applying it to the core or added together to the core in one or more coating applications in a revolving pan containing the cores. Generally, the coating syrup is very hot, about 130° F. to 200° F., and the flavor may volatilize if preblended with the coating syrup too early.

The concentrated coating syrup is applied to the gum cores as a hot liquid, the sugar or polyol allowed to crystallize, and the coating then dried with warm, dry air. This is repeated in about 30 to 100 applications to obtain a hard shell coated product having an increased weight gain of about 40% to 75%. A flavor is applied with one, two, three or even four or more of these coating applications. Each time flavor is added, several non-flavored coatings are applied to cover the flavor before the next flavor coat is applied. This reduces volatilization of the flavor during the coating process.

### EXAMPLES

The following examples of the invention and comparative examples are provided by way of explanation and illustration.

### Example 1-6

Varenicline tartrate can be used in the coating formula on the various pellet gum formulations. The following Table 1 shows some sugar type formulas:

TABLE 1
Varenicline Coating Formulations (Values are in weight %)

| | EX. 1 | EX. 2 | EX. 3 | EX. 4 | EX. 5 | EX. 6 |
|---|---|---|---|---|---|---|
| Sucrose | 97 | 95.2 | 93.6 | 96.8 | 94.9 | 93.1 |
| Gum Arabic | 2 | 3 | 4 | 2 | 3 | 4 |
| Titanium | 0.5 | 1 | 1 | — | — | — |
| dioxide | | | | | | |
| Calcium | — | — | — | 0.5 | 1 | 3 |
| carbonate | | | | | | |
| Flavor | 0.3 | 0.5 | 0.8 | 0.5 | 0.8 | 0.3 |
| Wax | 0.1 | 0.1 | 0.1 | 0.1 | 0.1 | 0.1 |
| Varenicline | 0.1 | 0.2 | 0.5 | 0.1 | 0.2 | 0.5 |
| tartrate | | | | | | |

The above formulations are made by making a syrup by dissolving the sugar and gum arabic in solution at about 75% solids at boiling, and suspending titanium dioxide or calcium carbonate in this syrup. Varenicline tartrate may be dissolved in water, not mixed with hot syrup, but added between coatings, or it may be added to the hot syrup and used in the early stages of coating or used throughout the coating process.

Flavor is not mixed with the hot syrup, but added at low levels with one or more coats. Varenicline tartrate may be dissolved in flavor and added to the coating. After the final coats are applied and dried, wax is applied to give a smooth polish. A 1.5 gm piece of gum containing 0.5 w/w % Varenicline tartrate contains 4.4 mg of the drug as the free base.

### Examples 7-10

Varenicline tartrate may also be used in coating of sugarless gum centers. Like sugar gum centers, the base formulation can be increased in proportion to the amount of coating applied to the center. Formulations with and without varenicline tartrate for low and high moisture gum can be used to make gum centers. Generally, the base level may be increased to 30-46% with the other ingredients proportionally reduced. Some typical gum formulas are in Table 2.

TABLE 2
(Values are in weight %)

| | EX. 7 | EX. 8 | EX. 9 | EX. 10 |
|---|---|---|---|---|
| Gum base | 35 | 35 | 30 | 30 |
| Calcium carbonate | | | 5 | 10 |
| Sorbitol | 43.3 | 45 | 45.9 | 40.3 |
| Mannitol | 10 | 10 | 5 | 10 |
| Glycerin | | 8 | 2 | |
| Sorbitol Liquid | 10 | | 10 | 8 |
| Flavor | 1.4 | 1.6 | 1.4 | 1.3 |
| Aspartame | 0.2 | 0.2 | 0.2 | 0.3 |
| Varenicline tartrate | 0.1 | 0.2 | 0.5 | 0.1 |

In the above center formulations, the high intensity sweetener used is aspartame. However other high intensity sweeteners such as alitame, acesulfame K, salts of acesulfame, cyclamate and its salts, saccharin and its salts, neotame, sucralose, thaurnatin, monellin, dihydrochalcone, stevioside, glycyrrhizin and combinations thereof may be used in any of the examples with the level adjusted for sweetness.

Lycasin and other polyols such as maltitol, xylitol, lactitol and hydrogenated isomaltulose may also be used in the gum center formulations at various levels. The texture may be adjusted by varying glycerin or sorbitol liquid. Sweetness of the center formulation can also be adjusted by varying the level of high intensity sweetener. A 1.5 gm piece of gum containing 0.2 w/w % varenicline tartrate contains 1.7 mg of the drug as the free base.

1. A chewing gum composition comprising a water insoluble base portion; a water soluble portion combined with said water insoluble base portion; and an effective amount of varenicline or its pharmaceutically acceptable salt combined with said water insoluble base portion and said water soluble portion to form said chewing gum composition.

2. The chewing gum composition according to claim 1, wherein said water insoluble base portion includes ingredients selected from the group consisting of elastomers, resins, fats, oils, softeners, inorganic fillers, organic fillers, wax, elastomer plasticizers, texturizers, emulsifiers, colorants, whiteners, and combinations thereof.

3. The chewing gum composition according to claim 1, wherein said water soluble portion includes ingredients selected from the group consisting of sweeteners, bulk sweeteners, high intensity sweeteners, low caloric bulking agents, bulking agents, flavoring agents, softeners, emulsifiers, colorants, acidulants, fillers, antioxidants, medicaments, and combinations thereof.

4. The chewing gum composition according to claim 1, wherein said varenicline or its pharmaceutical acceptable salt includes about 0.1 mg to about 10 mg of varenicline tartrate.

5. The chewing gum composition according to claim 1, wherein said varenicline or its pharmaceutical acceptable salt comprises particles having a particle size of about 0.1 microns to about 200 microns.

6. The chewing gum composition according to claim 1, wherein the chewing gum composition is in a form selected from the group consisting of a single piece, a stick, a slab, a pellet, a ball, any unitary structure, and over-coated formulation enclosing a gum center.

7. A method for reducing nicotine addiction and aiding in the cessation or lessening of tobacco use in an individual, comprising the steps of administering to an oral cavity of an individual a chewing gum composition comprising an effective amount of an immediate release form of varenicline or its pharmaceutically acceptable salt; and chewing the chewing gum composition to cause the varenicline or its pharmaceutically acceptable salt to be released from the chewing gum composition into the oral cavity of the individual.

8. The method according to claim 7, wherein said chewing step is defined as chewing for at least two minutes.

9. The method according to claim 8, wherein said chewing step is defined as chewing for at least thirty minutes

10. The method according to claim 9, wherein said chewing step is defined as chewing for at least sixty minutes.

11. A method of manufacturing a chewing gum composition comprising a therapeutically effective amount of varenicline tartrate comprising the steps of:
grinding varenicline tartrate into a fine powder having a particle size of approximately 0.1 microns to about 200 microns;
mixing the varenicline tartrate powder with a liquid to form an incorporation vehicle; and
mixing the incorporation vehicle with a chewing gum carrier to form a chewing gum composition.

12. The chewing gum composition according to claim 11, further including the step of coating the chewing gum composition with a coating including varenicline tartrate.

13. A chewing gum composition comprising a compressible excipient tablet; and a coating including an effective amount of varenicline or its pharmaceutically acceptable salt thereof, wherein said coating substantially surrounds said compressible excipient tablet.

14. A chewing gum composition comprising a compressible excipient tablet containing an effective amount of varenicline or its pharmaceutically acceptable salt thereof, and a chewing gum coating, wherein said coating substantially surrounds said compressible excipient tablet.

15. The chewing gum composition according to claim 13, wherein said compressible excipient tablet includes ingredients selected from the group consisting of dextrose, sucrose, saccharides, sorbitol, mannitol, isomalitol, compressible sugar alcohols, elastomers, resins, fats, softeners, inorganic fillers, organic fillers, waxes, elastomer plasticizers, texturizers, emulsifiers, colorants, whiteners, sweeteners, bulk sweeteners, high intensity sweeteners, low caloric bulking agents, bulking agents, flavoring agents, softeners, colorants, acidulants, antioxidants, medicaments, and combinations thereof.
